(19) United States
(12) Patent Application Publication
(10) Pub. No.: US 2022/0319662 A1
(43) Pub. Date: Oct. 6, 2022

(54) REHABILITATION WORK SUPPORT APPARATUS, REHABILITATION WORK SUPPORT SYSTEM, REHABILITATION WORK SUPPORT METHOD, AND COMPUTER READABLE MEDIUM

(71) Applicant: NEC Corporation, Minato-ku, Tokyo (JP)

(72) Inventors: Yuki Kosaka, Tokyo (JP); Toshinori Hosoi, Tokyo (JP); Masahiro Kubo, Tokyo (JP); Yuan Luo, Tokyo (JP); Shigemi Kitahara, Tokyo (JP)

(73) Assignee: NEC Corporation, Minato-ku, Tokyo (JP)

(21) Appl. No.: 17/764,692

(22) PCT Filed: Jul. 29, 2020
(86) PCT No.: PCT/JP2020/029107

§ 371 Date: Mar. 29, 2022

(30) Foreign Application Priority Data
Oct. 4, 2019 (JP) 2019-184151

(51) Int. Cl. G16H 20/30; G16H 10/60; A61B 5/00

(52) U.S. Cl. CPC G16H 20/30; G16H 10/60; A61B 5/7435; A61B 5/746; A61B 2505/09

(57) ABSTRACT

A rehabilitation work support apparatus, a rehabilitation work support method, and a program capable of efficiently managing patient's actual current ability values are provided. A rehabilitation work support apparatus (1) includes a change prediction unit (2) that predicts whether or not an actual current ability value of a patient during a rehabilitation period has changed from a latest ability value of that patient stored in a storage device based on an elapsed time from a time of an evaluation of the latest ability value of the patient stored in the storage device, and a change prediction output unit (3) that performs control so as to output a result of the prediction made by the change prediction unit (2).

### TECHNICAL FIELD

The present invention relates to a rehabilitation work support apparatus, a rehabilitation work support method, and a program.

### BACKGROUND ART

In recent years, there has been a need for a technology for supporting rehabilitation (e.g., a rehabilitation training or a rehabilitation therapy) (hereinafter also referred to as “rehab”), and research and development for such technology has been pursued.

For example, Patent Literature 1 discloses a rehabilitation management apparatus that enables both a patient and a physical therapist to recognize the effect of rehabilitation. This rehabilitation management apparatus generates evaluation data including the degree of recovery of the patient and the like based on rehabilitation performance data and rehabilitation plan data.

### CITATION LIST

### Patent Literature

Patent Literature 1: Japanese Unexamined Patent Application Publication No. 2013-161315

### SUMMARY OF INVENTION

### Technical Problem

In the rehabilitation management apparatus disclosed in Patent Literature 1, the degree of recovery of a patient to be evaluated is estimated by referring to the degrees of recoveries of past patients who are similar to the patient to be evaluated. However, for example, as in the case of evaluations in an FIM (Function Independence Measure), in some cases, it is necessary to observe the actual movements of the patient to evaluate the patient's ability values. In such cases, data on the patient will not be updated unless a therapist such as a physical therapist observes the actual movements of the patient. However, since the ability values may not have changed after the previous evaluation, it is inefficient to evaluate the ability value constantly (e.g., on a daily basis).

Therefore, one of the objects to be attained by example embodiments disclosed in this specification is to provide a rehabilitation work support apparatus, a rehabilitation work support method, and a program capable of efficiently managing patient's actual current ability values.

### Solution to Problem

A rehabilitation work support apparatus according to a first aspect of the present disclosure includes:

change prediction means for predicting whether or not an actual current ability value of a patient during a rehabilitation period has changed from a latest ability value of that patient stored in a storage device based on an elapsed time from a time of an evaluation of the latest ability value of the patient stored in the storage device; and

change prediction output means for performing control so as to output a result of the prediction made by the change prediction means.

A rehabilitation work support method according to a second aspect of the present disclosure includes:

predicting whether or not an actual current ability value of a patient during a rehabilitation period has changed from a latest ability value of that patient stored in a storage device based on an elapsed time from a time of an evaluation of the latest ability value of the patient stored in the storage device; and

performing control so as to output a result of the prediction.

A program according to a third aspect of the present disclosure causes a computer to perform:

a change prediction step of predicting whether or not an actual current ability value of a patient during a rehabilitation period has changed from a latest ability value of that patient stored in a storage device based on an elapsed time from a time of an evaluation of the latest ability value of the patient stored in the storage device; and

a change prediction output step of performing control so as to output a result of the prediction made in the change prediction step.

### Advantageous Effects of Invention

According to the present disclosure, it is possible to provide a rehabilitation work support apparatus, a rehabilitation work support method, and a program capable of efficiently managing patient's actual current ability values.

### BRIEF DESCRIPTION OF DRAWINGS

FIG. 1 is a block diagram showing an example of a configuration of a rehabilitation work support apparatus according to an outline of an example embodiment;

FIG. 2 is a block diagram showing an example of a configuration of a rehabilitation work support system according to an example embodiment;

FIG. 3A is a schematic diagram showing an example of a screen for selecting a task (a superordinate task) that a patient desires to accomplish through rehabilitation;

FIG. 3B is a schematic diagram for explaining an example of a screen for selecting a task (a subordinate task) that a patient desires to accomplish through rehabilitation;

FIG. 3C is a schematic diagram for showing an example of a screen for selecting an exercise item (a program) for accomplishing a task;

FIG. 4 is a table showing an example of past patient information used in a survival analysis;

FIG. 5 is a table showing an example of past patient information used to predict an ability value after a change;

FIG. 6 shows a display example of information representing a result of a prediction made by a change prediction unit, displayed in a portable terminal device;

FIG. 7 shows a display example of information representing an un-updated period for each patient displayed in a non-portable terminal device;

FIG. 8 is a schematic diagram showing an example of a hardware configuration of a rehabilitation work support apparatus according to an example embodiment;

FIG. 9 is a flowchart showing an example of an outputting operation related to a prediction of a change in an ability value, performed by a rehabilitation work support apparatus according to an example embodiment; and

FIG. 10 is a flowchart showing an example of an outputting operation related to an un-updated period, performed by a rehabilitation work support apparatus according to an example embodiment.

### DESCRIPTION OF EMBODIMENTS

### Overview of Example Embodiment

Prior to describing an example embodiment in detail, an outline of the example embodiment will be described.

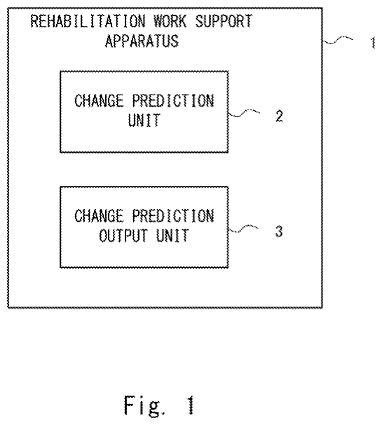

FIG. 1 is a block diagram showing an example of a configuration of a rehabilitation work support apparatus 1 according to an outline of an example embodiment. As shown in FIG. 1, the rehabilitation work support apparatus 1 includes a change prediction unit 2 and a change prediction output unit 3.

The change prediction unit 2 predicts whether or not an actual current ability value of a patient during a rehabilitation period has changed from a latest ability value of that patient stored in a storage device based on the elapsed time from the time of the evaluation of the latest ability value of the patient stored in the storage device.

Note that the ability value is an ability value related to patient's activities in daily life, such as ADL (Activities of Daily Living) or IADL (Instrumental Activities of Daily Living). The change prediction unit 2 may make a prediction for each type of ability, i.e., for each type of patient's activity in daily life. For example, the change prediction unit 2 may make a prediction whether or not an ability value for eating movements has changed, whether or not an ability value for toilet movements has changed, and the like.

The change prediction output unit 3 performs control so as to output the results of the predictions made by the change prediction unit 2. For example, the change prediction output unit 3 outputs the prediction results to other apparatuses (e.g., a terminal device). As a result, the prediction results are displayed on a display of the other apparatuses. Note that the change prediction output unit 3 may perform control so as to display the prediction results on a display provided in the rehabilitation work support apparatus 1.

When a prediction result indicating that an actual current ability value of a patient has changed from a latest ability value of that patient stored in the storage device is obtained, a therapist may evaluate the ability value of the patient and update data stored in the storage device. That is, according to the rehabilitation work support apparatus 1, the therapist can determine whether or not it is necessary to re-evaluate the patient's ability value at the present time by checking the output from the rehabilitation work support apparatus 1. Since the therapist can know an appropriate timing for making an evaluation of an ability value as described above, he/she does not need to evaluate the ability value constantly (e.g., on a daily basis) in order to prevent the deviation of the actual ability value from the recorded ability value. Therefore, according to the rehabilitation work support apparatus 1, it is possible to efficiently manage patient's actual current ability values.

### Details of Example Embodiment

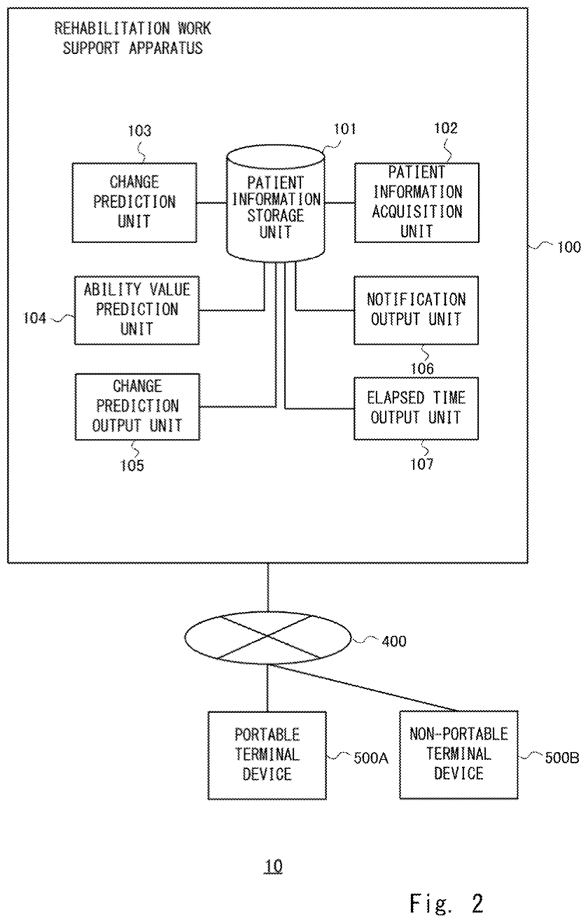

An example embodiment according to the present invention will be described hereinafter with reference to the drawings. FIG. 2 is a block diagram showing an example of a configuration of a rehabilitation work support system 10 according to an example embodiment.

The rehabilitation work support system 10 includes a rehabilitation work support apparatus 100, a portable terminal device 500A, and a non-portable terminal device 500B. In the following description, when the portable terminal device 500A and the non-portable terminal device 500B are mentioned without distinguishing them from each other, they may be referred to as terminal devices 500. The rehabilitation work support apparatus 100 is connected to the terminal devices 500 through a network 400 wirelessly or through a cable.

The rehabilitation work support apparatus 100 is configured, for example, as a server. Further, the portable terminal device 500A is an arbitrary portable terminal device such as a tablet terminal or a smartphone. Further, the non-portable terminal device 500B is a stationary terminal device such as a personal computer. The terminal device 500 is equipped with an input device and an output device, and hence is able to receive information to be transmitted to the rehabilitation work support apparatus 100 and output (display) information received from the rehabilitation work support apparatus 100.

Note that, in the example shown in FIG. 2, the rehabilitation work support system 10 includes both the portable terminal device 500A and the non-portable terminal device 500B, but it may include only one of them. Further, the numbers of portable terminal devices 500A and non-portable terminal devices 500B is not limited to the numbers shown in FIG. 2.

The rehabilitation work support apparatus 100 is an apparatus for supporting the work of a therapist who performs a rehabilitation therapy for a patient. For example, the therapist works at a certain facility such as a rehabilitation hospital and carries the portable terminal device 500A. Further, the non-portable terminal device 500B is installed, for example, in the certain facility, and the therapist can also use the non-portable terminal device 500B.

As shown in FIG. 2, the rehabilitation work support apparatus 100 includes a patient information storage unit 101, a patient information acquisition unit 102, a change prediction unit 103, an ability value prediction unit 104, a change prediction output unit 105, a notification output unit 106, and an elapsed time output unit 107.

The patient information storage unit 101 corresponds to the aforementioned storage device and stores patient information. The patient information includes patient information for a target patient (target patient information) and patient information for past patients (past patient information). Note that the target patient is a patient who will start rehabilitation or is presently performing rehabilitation. The past patient is a patient who performed rehabilitation in the past.

The patient information is information about a patient. Specifically, for example, the patient information includes data items such as patient's attributes, patient's symptoms, ability value information, contents and the number of rehabilitation sessions performed in the past, and information about a therapist in charge. However, these data items are merely examples, and the patient information is not limited to them. Note that, in the target patient information, some of these data items may have NULL values (null values) because data in them are not determined. For example, regarding the contents of performed rehabilitation, it is a data item that will be determined after the rehabilitation is performed, so this data item has a NULL value before the rehabilitation is performed. Data in each data item in the patient information is, for example, expressed by a numerical code.

Although the rehabilitation work support apparatus 100 includes the patient information storage unit 101 in this example embodiment, the patient information storage unit 101 may be implemented by an external apparatus. In such a case, the rehabilitation work support apparatus 100 may acquire the patient information from this external apparatus.

The attributes of the patient include, specifically, an arbitrary attribute information such as the gender, the age, and the like of the patient. The ability value is an ability value related to patient's activities in daily life as described above, and is, for example, an ability value related to the ADL or the IADL.

In this example embodiment, the ability value is, specifically, an evaluation score for each evaluation item in an FIM (Function Independence Measure) table. The evaluation item corresponds to the type of ability. For example, in the FIM, evaluations are made for 18 different items. In this case, the patient information includes data on ability values (i.e., evaluation scores in the FIM) for the 18 types of abilities. The evaluation scores in the FIM are determined by a user (e.g., a therapist) as he/she observes how much assistance is required for certain activities in daily life. Therefore, it is necessary to observe the patient in order to update ability values contained in the patient information stored in the patient information storage unit 101. Note that although the FIM is used as an example of ability values in this example embodiment, other ability values may also be used. Further, although the ability value is a value for each of a plurality of types of abilities (a value for each FIM item) in this example embodiment, the ability value may be an ability value for only one type of ability.

The ability value information includes patient's ability values at various times (e.g., ability values when the patient is admitted to a hospital, ability values during the hospitalization, and ability values when the patient is discharged from the hospital), target values for the patient's ability value in rehabilitation, and differences (gaps) between ability values at a certain time and their target values. Further, in particular, the ability value information include data about a change in an ability value in this example embodiment. Specifically, the data about a change in an ability value is information about, for each type of ability (for each item in the FIM), an ability value before a change, an ability value after the change, and the number of days that have been taken before the change occurs. Note that as the number of days that have been taken before the change occurs can be, for example, the number of days that have been taken before the ability value stored in the patient information storage unit 101 is updated can be used.

The contents of rehabilitation include, for example, tasks that the patient desires to accomplish through the rehabilitation and contents (programs) of practices for accomplishing the tasks. Regarding the tasks, a superordinate task(s) and a subordinate task(s) may be set.

The patient information acquisition unit 102 acquires the above-described patient information and stores the acquired patient information in the patient information storage unit 101. In this example embodiment, the patient information acquisition unit 102 provides a GUI (Graphical User Interface) to the terminal device 500 and acquires patient information entered to the terminal device 500. For example, when the user (the therapist) has newly evaluated a patient's ability value, he/she registers the evaluated value in the rehabilitation work support apparatus 100 by using the terminal device 500.

Further, in particular, the patient information acquisition unit 102 specifies contents of rehabilitation performed by the patient by receiving a choice(s) selected from a plurality of choices in regard to the contents of the rehabilitation from the user (the therapist). Here, recording of contents of commonly practiced rehabilitation will be discussed. In general, contents of rehabilitation are recorded by describing them in a free format. In such a case, even if the contents of rehabilitation sessions are the same as each other, it is difficult to manage information about them as the contents of the same rehabilitation sessions because their descriptions are different from each other. That is, it is difficult to analyze the data. In contrast, in this example embodiment, as described above, the contents of rehabilitation are specified by receiving a choice selected from a plurality of choices prepared in advance, so the data can be acquired in a format that can be easily handled in data processing.

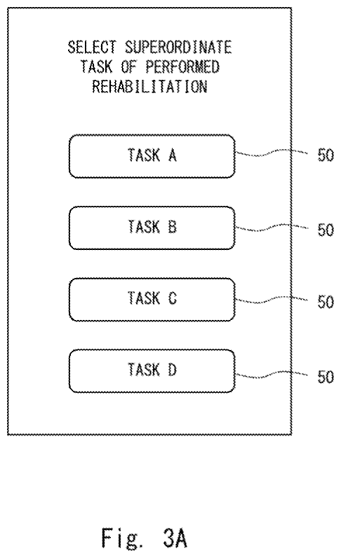

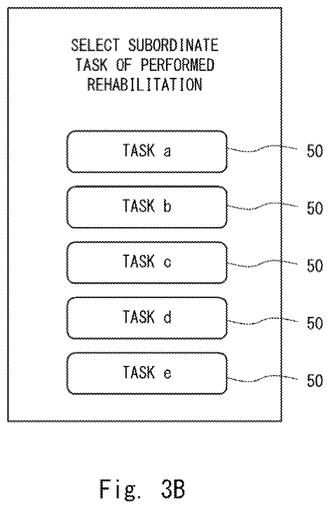

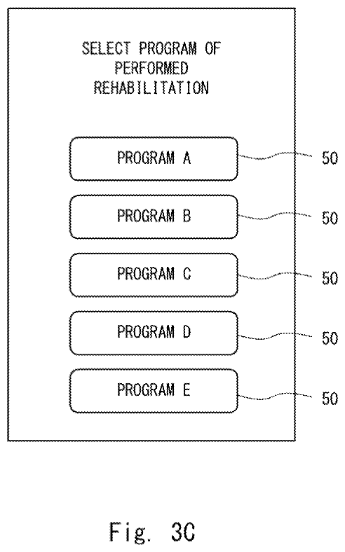

FIGS. 3A to 3C are schematic diagrams showing examples of screens (e.g., windows) that are displayed on the display of the portable terminal device 500A (e.g., the smartphone) when the patient information acquisition unit 102 acquires the contents of rehabilitation. Specifically, FIG. 3A is a schematic diagram showing an example of a screen (e.g., a window) for selecting a task (a superordinate task) that the patient desires to accomplish through the rehabilitation. FIG. 3B is a schematic diagram showing an example of a screen for selecting a task (a subordinate task) that the patient desires to accomplish through the rehabilitation. FIG. 3C is a schematic diagram showing an example of a screen for selecting contents (a program) of a practice for accomplishing a task.

The therapist selects, among the choices 50, a choice that represents the contents of the rehabilitation performed by the patient. Note that the user may be able to make a more detailed selection when he/she specify contents (a program) of a practice. For example, contents (a program) of a practice may be specified by selecting an item for each of the posture of the patient, a part of the body for which the rehabilitation is performed, and contents of an exercise. Further, other information, in addition to the contents of the rehabilitation, may also be specified by receiving a choice selected from a plurality of choices. The patient information acquisition unit 102 can also acquire patient information from the non-portable terminal device 500B, and similar screens (i.e., similar windows) may be displayed on the non-portable terminal device 500B. As described above, in this example embodiment, the patient information includes information specified as described above.

The change prediction unit 103 corresponds to the change prediction unit 2 shown in FIG. 1. The change prediction unit 103 predicts whether or not an actual current ability value of a patient during a rehabilitation period has changed from a latest ability value of that patient stored in the patient information storage unit 101.

In this example embodiment, the change prediction unit 103 performs the prediction based on the elapsed time from the time of the evaluation of the latest ability value of the patient stored in the patient information storage unit 101 and features of the patient. Note that as the elapsed time from the time of the evaluation of the latest ability value, for example, an elapsed time from the time of the update of the ability value stored in the patient information storage unit 101 can be used. However, other times may be used as the elapsed time from the time of the evaluation of the latest ability value. For example, when the patient information includes a date on which the evaluation was made, the elapsed time from this date may be used.

A prediction process performed in the change prediction unit 103 in this example embodiment will be described hereinafter in detail.

In this example embodiment, the change prediction unit 103 predicts whether or not an actual current ability value of a target patient has changed from a latest ability value thereof stored in the patient information storage unit 101 by performing a survival analysis. Specifically, a change in an ability value of the target patient from the latest ability value thereof stored in the patient information storage unit 101 is defined as an event in the survival analysis. Then, the change prediction unit 103 calculates, for a target patient u having a feature represented by a feature value Xᵤ, a probability P(t, Xᵤ) that the above-defined event will not occur in a time t. This probability P(t, Xᵤ) corresponds to a survival probability in the survival analysis. That is, the change prediction unit 103 makes a prediction by performing a survival analysis in which a probability that an actual ability value of a patient having a certain feature at a specific time has not changed from a latest ability value of that patient stored in the storage device is defined as the survival probability. In other words, the change prediction unit 103 makes a prediction by performing a survival analysis in which a probability that the actual ability value is equal to the stored latest ability value is defined as the survival probability. Note that the specific time is the aforementioned time t, and specifically is a time that represents an elapsed time from the time of the evaluation of the latest ability value of the patient stored in the patient information storage unit 101. Note that, for example, a Kaplan-Meier method may be used for the survival analysis.

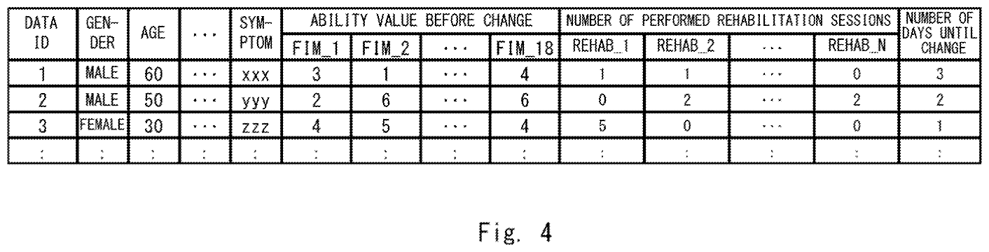

The survival analysis is performed by using statistical data of past patient information stored in the patient information storage unit 101. FIG. 4 is a table showing an example of the past patient information used for the survival analysis. Note that, in FIG. 4, only items that used to calculate the survival probability in this example embodiment are extracted from all the items included in the past patient information and are shown as examples. FIG. 4 shows, for example, data on changes in ability values in some items (e.g., toilet movements) in the FIM. Note that, for other items in the FIM, similar past patient information is also stored in the patient information storage unit 101.

In FIG. 4, the past patient information includes feature values representing patient's conditions immediately before an ability value for a certain item in the FIM changes, and the number of days until the change. More specifically, in the example shown in FIG. 4, as the feature values representing patient's conditions immediately before an ability value for a certain item in the FIM changes, the gender, the age, symptoms, an ability value in each item in the FIM before the change, and the number of rehabilitation sessions performed before the occurrence of the change are shown. Note that the ability value in each item in the FIM before the change is an ability value in each of some items in the FIM immediately before the update of the ability value in the certain item in the FIM (i.e., is the latest ability value of the patient in each item already stored in the patient information storage unit 101 at the time of the update). That is, assuming that the certain item in the FIM is toilet movements, the ability value in each item in the FIM before the change indicates the latest ability value of the patient in each item that is already stored in the patient information storage unit 101 at the time of the update of the ability value of the toilet movements. Further, the number of rehabilitation sessions performed before the occurrence of the change is the number of rehabilitation sessions counted for each content of the rehabilitation. That is, assuming that the certain item in the FIM is toilet movements, the number of rehabilitation sessions performed before the occurrence of the change indicates which content of the rehabilitation has been performed by the patient and to what extent that content has been performed before the ability value of toilet movements is updated. In FIG. 4, for each of N types of contents in the rehabilitation, which content of the rehabilitation has been performed and how many times the content has been performed are shown.

Note that, for the same patient, a change in an ability value in a certain item in the FIM may occur a plurality of times during the rehabilitation period. Therefore, a group of data shown in FIG. 4 may include a plurality of data for the same patient.

The change prediction unit 103 performs a survival analysis by using, among data on changes in ability values in the FIM item to be predicted like those shown in FIG. 4, data including feature values similar to those of a patient(s) for whom the prediction is made, and thereby calculates a survival probability for the patient in this FIM item.

When the calculated survival probability is lower than a predetermined threshold value (e.g., 0.5), the change prediction unit 103 predicts that the actual value of the patient in this FIM item has deviated from the value thereof stored in the patient information storage unit 101. That is, the change prediction unit 103 predicts that the actual ability value has changed from the latest ability value of that patient stored in the patient information storage unit 101. On the other hand, when the calculated survival probability is equal or higher than the predetermined threshold value (e.g., 0.5), the change prediction unit 103 predicts that the actual ability value for the FIM item has not changed from the latest ability value of that patient stored in the patient information storage unit 101.

In this example embodiment, the change prediction unit 103 makes a prediction for each type of ability as described above. Therefore, the user can know not only for which patient(s) an ability value(s) needs to be re-evaluated, but also for which ability(ies) the ability value(s) needs to be re-evaluated. However, the change prediction unit 103 may make predictions for all the types of abilities in a collective manner. In such a case, the survival analysis may be performed by using, among the data on changes in ability values in all the FIM items, data including feature values similar to those of the patient for whom the prediction is made.

Further, in this example embodiment, as described above, the change prediction unit 103 makes a prediction by calculating a survival probability according to the features of the patient for which the prediction is made. In particular, the features include the contents of the rehabilitation performed by the patient. Therefore, similarities between the rehabilitation history of past patients and the rehabilitation history of the patient for which the prediction is made are taken into consideration, thus making it possible to make a prediction that is more accurate than that in the case where such features are not used. Further, the features also include patient's latest ability values stored in the patient information storage unit 101. Therefore, similarities between the ability values of past patients and those of the patient for which the prediction is made are taken into consideration, thus making it possible to make a prediction that is more accurate than that in the case where such features are not used.

Note that the change prediction unit 103 may make a prediction without using the features of the patient, or may make a prediction by using only some of the feature values shown in FIG. 4.

The change prediction unit 103 makes the above-described prediction for all the types of abilities of all the target patients, for example, at regular intervals. However, the change prediction unit 103 may make the prediction for some of the target patients, or may make the prediction for some types of abilities. The change prediction unit 103 stores the results of the predictions in the patient information storage unit 101.

The ability value prediction unit 104 predicts an ability value after a change. In this example embodiment, the ability value prediction unit 104 predicts, for a patient of which the change prediction unit 103 has predicted that an actual ability value has changed from a latest ability value thereof stored in the patient information storage unit 101, an ability value after the change.

The ability value prediction unit 104 predicts the ability value after the change by inputting information about the patient, who is in the rehabilitation period, to a prediction model using information about a plurality of patients in the past (hereinafter also referred to as a plurality of pieces of past patient information). Specifically, the ability value prediction unit 104 predicts the ability value of the target patient after the change by using a prediction model using data including ability values of past patients for the ability to be predicted that are obtained before a change and ability values thereof that are obtained after the change.

A prediction of an ability value after a change, made by the ability value prediction unit 104 in this example embodiment will be described hereinafter in detail.

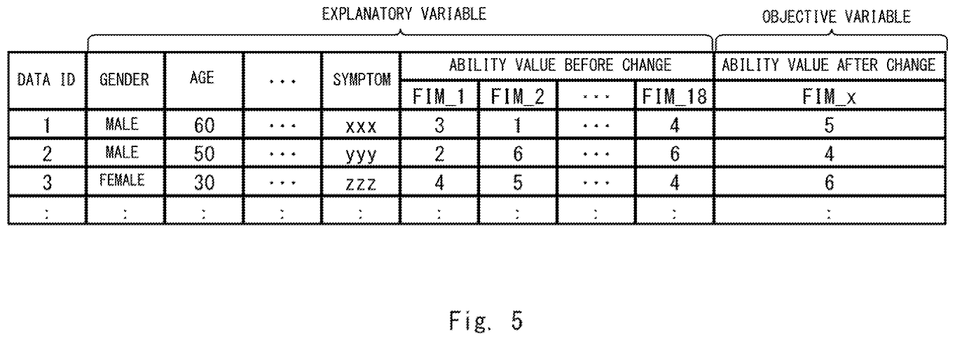

FIG. 5 is a table showing an example of past patient information used to predict an ability value after a change. Note that, In FIG. 5, only items that used to predict the ability value after the change in this example embodiment are extracted from all the items included in the past patient information and are shown as examples.

In this example embodiment, for example, the ability value prediction unit 104 performs a prediction as follows. That is, the ability value prediction unit 104 makes a prediction by using a linear regression model in which patient information representing patient's conditions before a change is used as explanatory variables and after-change ability values to be predicted are used as objective variables. Note that the parameters of the linear regression model can be determined by applying a known method such as a least-squares method to the past patient information. The patient information representing patient's conditions before the change includes at least an ability value for an ability to be predicted before the change, and may also include patient's conditions such as an ability value(s) for an ability(ies) other than the ability to be predicted before the change, the gender, the age, and symptoms. The ability value prediction unit 104 inputs information used as the explanatory variables (patient information representing the patient's conditions before the change) included in the target patient information into the linear regression model in which parameter values have already been learned, and thereby obtains the result of the prediction of the ability value after the change.

Note that, in this example embodiment, the ability value prediction unit 104 makes, as an example, a prediction using a linear regression model as a prediction model. However, the prediction model is not limited to such models and may be an arbitrary machine learning model for solving a regression problem. For example, Support vector regression may be used as the prediction model.

The ability value prediction unit 104 stores the calculated predicted value in the patient information storage unit 101.

As described above, in this example embodiment, since the ability value prediction unit 104 calculates the predicted value of the ability value after the change, it is possible to present, to a user, the ability value after the change as well as the information indicating the occurrence of the change in the ability value.

The change prediction output unit 105 corresponds to the change prediction output unit 3 shown in FIG. 1 and performs control so as to output the result of the prediction made by the change prediction unit 103 to the terminal device 500. In this example embodiment, the change prediction output unit 105 outputs, as the prediction result, information indicating, for which ability, the ability value has changed. That is, the change prediction output unit 105 outputs information indicating, for which ability and for which patient, the actual current ability value has changed from the latest ability value of that patient stored in the patient information storage unit 101. Further, the change prediction output unit 105 performs control so as to output the ability value after the change predicted by the ability value prediction unit 104 to the terminal device 500.

For example, when an application running on the terminal device 500 requests such information, the change prediction output unit 105 refers to the patient information storage unit 101 and transmits the results of the predictions made by the change prediction unit 103 and the ability value prediction unit 104 to the terminal device 500. Note that the change prediction output unit 105 may transmit only the result of the prediction made by the change prediction unit 103 to the terminal device 500.

Specifically, for example, when a user such as a therapist provides an input for instructing to display such information to an application running on the terminal device 500, the request for the information is transmitted from the terminal device 500 to the rehabilitation work support apparatus 100. As a result, the application running on the terminal device 500 displays the information received from the rehabilitation work support apparatus 100 on the display of the terminal device 500. Note that the output (the display) of the prediction results of the change prediction unit 103 and the ability value prediction unit 104 may be performed without a request from the terminal device 500.

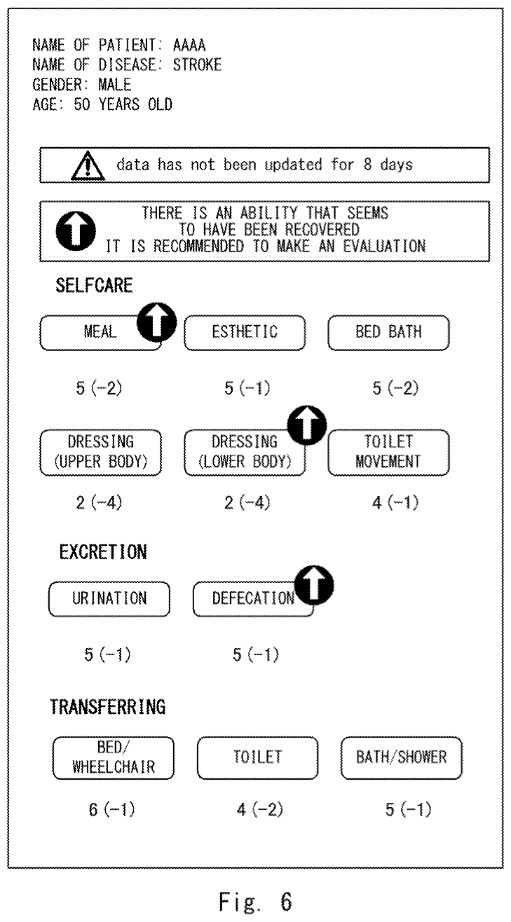

FIG. 6 shows a display example of information representing the prediction result of the change prediction unit 103, displayed on the portable terminal device 500A. In the example shown in FIG. 6, certain marks (arrows in the example shown in FIG. 6) are added to abilities of which the actual current ability values of the patient are predicted to have changed from the latest ability values thereof stored in the patient information storage unit 101. Note that, in the example shown in FIG. 6, for each type of ability, a numerical value representing a latest ability value stored in the patient information storage unit 101 and that representing a difference between a target value and the latest ability value are shown. More specifically, in FIG. 6, the differences are shown in parentheses. As described above, for example, an ability(ies) of which the ability value(s) is predicted to have changed is specified in a screen (e.g., a window) showing a list of abilities.

Note that, as shown in FIG. 6, patient information other than the ability values may be displayed along with the information about the ability values, and/or a warning message or the like for the user may be displayed. Further, values predicted by the ability value prediction unit 104 may also be displayed. Note that although FIG. 6 is shown as an example of a display shown in the portable terminal device 500A, a display like the one shown in FIG. 6 may be displayed in a non-portable terminal device 500B.

Further, the prediction results by the change prediction unit 103 and the ability value prediction unit 104 may be displayed as an electronic medical record, or may be displayed on an electronic bulletin board.

When an actual current ability value of the patient during the rehabilitation period is predicted to have changed from the latest ability value thereof stored in the patient information storage unit 101, the notification output unit 106 performs control so as to output a notification message for informing the therapist or the like of the prediction of the change. In this way, it is possible to make the therapist aware of the existence of such a patient. That is, it is possible to urge the therapist to re-evaluate the ability value, i.e., to update the ability value stored in the patient information storage unit 101.

Specifically, for example, the notification output unit 106 transmits a notification message to the terminal device 500. In the terminal device 500, the received notification message is displayed on the display. Note that, in the terminal device 500, the notification message may be output in the form of a sound (e.g., a voice).

Further, the notification output unit 106 may output a notification message at a predetermined notification timing. For example, the notification output unit 106 may perform control so as to output a notification message at a timing corresponding to a timing at which rehabilitation of the patient whose ability value is predicted to have changed is performed. Specifically, for example, a notification may be provided at a predetermined time on the day on which the rehabilitation by the target patient is performed. Further, the predetermined time may be, for example, a fixed time in the morning or evening, a time immediately before the start of the rehabilitation of the patient, or a time immediately after the end of the rehabilitation of the patient. In such a case, for example, the notification output unit 106 checks the timing at which rehabilitation of each patient is performed by referring to a database in which the schedule of the rehabilitation of each patient is managed, and determines the notification timing of the notification message. Since a notification is provided according to the timing at which the rehabilitation of the target patient is performed as described above, it is possible to send a notification at a desirable and appropriate timing.

Further, the notification output unit 106 may perform control so that when a predetermined operation has not been performed within a predetermined time after a notification message is output, it outputs the notification message again. For example, when an operation for checking the ability value of the target patient (specifically, for example, an operation for starting the aforementioned application) is not performed in the terminal device 500 to which a notification message has been sent, the notification output unit 106 outputs the notification message again. In this way, it is possible to urge more reliably the therapist to re-evaluate the ability value, i.e., to update the ability value stored in the patient information storage unit 101.

Further, the notification that the terminal device 500 has received from the rehabilitation work support apparatus 100 may be displayed thereon at a predetermined timing, such as when the non-portable terminal device 500B is logged in or when an application running on the portable terminal device 500A is started. Further, the notification and the displaying may be performed, among all the target patients, only for target patients whom the therapist is in charge of.

The elapsed time output unit 107 performs control so as to output, to the terminal device 500, information representing, for each patient, the elapsed time from the time of the last update of an ability value stored in the patient information storage unit 101. Note that the time of the last update means a time at which the ability value for any of the abilities of the patient was updated. For example, the elapsed time output unit 107 periodically calculates the elapsed time from the time of the last update. Specifically, for example, the elapsed time output unit 107 calculates the number of days elapsed from the last update date of the ability value based on the last update date of the ability value of each patient stored in the patient information storage unit 101 and the current date. That is, the elapsed time output unit 107 calculates a period during which the ability value in the patient information storage unit 101 has not been updated (hereinafter also referred to as an un-updated period). Then, the elapsed time output unit 107 performs control so as to output information indicating the calculated period (the un-updated period) to the terminal device 500. Note that the elapsed time output unit 107 may output the un-updated period while associating it with other information. For example, the elapsed time output unit 107 may output the un-updated period while associating it with information about the therapist in charge of the patient, the last update data, and the like.

When an application running on the terminal device 500 requests such information, the elapsed time output unit 107 outputs information indicating the calculated period to the terminal device 500. As a result, the application running on the terminal device 500 displays the information about the un-updated period for each patient on the display of the terminal device 500. Note that the output (the display) of the un-updated period may be performed without a request from the terminal device 500.

When the un-updated period has continued over a long period of time, there is a possibility that an actual current ability value of the patient has changed from the latest ability value thereof stored in the patient information storage unit 101. Therefore, by outputting the un-updated period, it is possible to find a patient(s) whose ability value has possibly changed. Therefore, it is possible to have an opportunity (or a trigger) for re-evaluating the ability value of the patient. According to this feature, it is sufficient if the re-evaluation is made only for a patient(s) whose ability value has possibly changed, thus making it possible to efficiently manage the actual current ability values of patients.

Note that, in this example embodiment, it is possible to have an opportunity (or a trigger) for making a re-evaluation based on the output by the change prediction output unit 105 as described above. Therefore, it can be considered that this example embodiment provides two mechanisms for urging a user or the like to re-evaluate an ability value(s). Therefore, it is also possible to configure (or construct) a rehabilitation work support apparatus having either a function of outputting a prediction made by the change prediction unit 103 or a function of outputting an un-updated period.

Note that when a therapist evaluates an ability value, he/she registers a new evaluated value in the rehabilitation work support apparatus 100. That is, in this case, the new evaluated value is acquired by the patient information acquisition unit 102, and the patient information stored in the patient information storage unit 101 is updated.

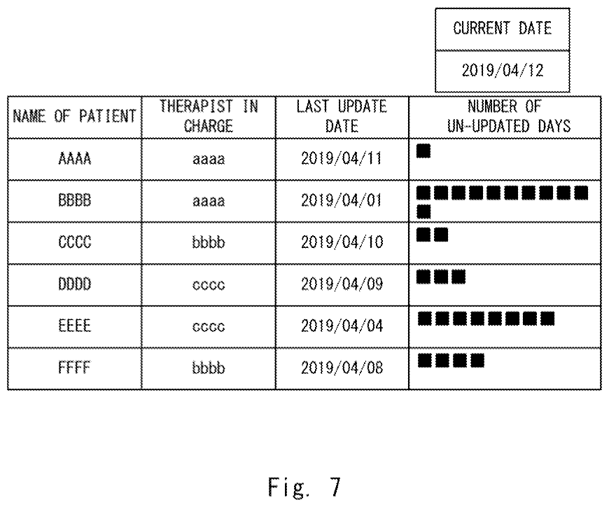

FIG. 7 shows a display example of information representing an un-updated period for each patient displayed on the non-portable terminal device 500B. In the example shown in FIG. 7, the number of un-updated days of an ability value in the patient information storage unit 101 is indicated by the number of rectangular marks so that it can be easily recognized visually. Note that the color of the marks may be changed according to the number of un-updated days of the ability value, so that it can be visually recognized more easily.

Further, in the example shown in FIG. 7, therapists in charge of respective patients are also displayed. Therefore, it is possible to check (i.e., find) a patient(s) for whom the ability values have not been re-evaluated for a long period of time, and a therapist(s) in charge of that patient(s). For example, an administrator can instruct a therapist in charge of such a patient to re-evaluate the ability values. Note that the displaying shown in FIG. 7 may be performed on the portable terminal device 500A. Further, as shown in FIG. 6, a warning message or the like indicating the number of un-updated days may be displayed.

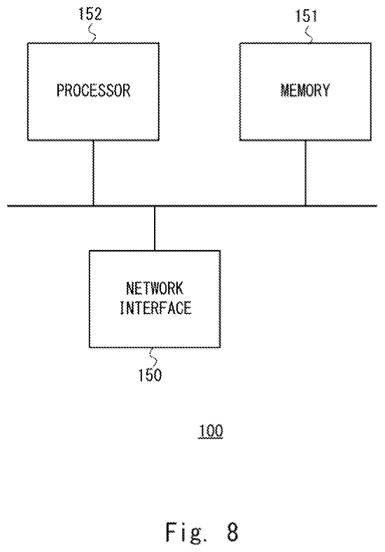

FIG. 8 is a schematic diagram showing an example of a hardware configuration of the rehabilitation work support apparatus 100. As shown in FIG. 8, the rehabilitation work support apparatus 100 includes a network interface 150, a memory 151, and a processor 152.

The network interface 150 is used to communicate with other arbitrary apparatus such as the terminal device 500.

The memory 151 is composed of, for example, a combination of a volatile memory and a non-volatile memory. The memory 151 is used to store software (a computer program) including one or more instructions executed by the processor 152, and data used for various processes performed by the rehabilitation work support apparatus 100. The patient information storage unit 101 shown in FIG. 2 is implemented, for example, by the memory 151, but may instead be implemented by other storage devices.

The processor 152 performs a process performed by each of the components shown in FIG. 2 by loading the software (the computer program) from the memory 151 and executing the loaded software. Specifically, the processor 152 performs processes performed by the patient information acquisition unit 102, the change prediction unit 103, the ability value prediction unit 104, the change prediction output unit 105, the notification output unit 106, and the elapsed time output unit 107.

The processor 152 may be, for example, a microprocessor, an MPU (Micro Processor Unit), or a CPU (Central Processing Unit). The processor 152 may include a plurality of processors.

As described above, the rehabilitation work support apparatus 100 has functions as a computer. Note that, similarly, the terminal device 500 has a hardware configuration like the one shown in FIG. 8. That is, the processes performed by the terminal device 500 are implemented, for example, by having the processor execute the program.

Further, the program may be stored in various types of non-transitory computer readable media and thereby supplied to computers. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Read Only Memory), CD-R, CD-R/W, and a semiconductor memory (such as a mask ROM, a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line (e.g., electric wires and optical fibers) or a wireless communication line.

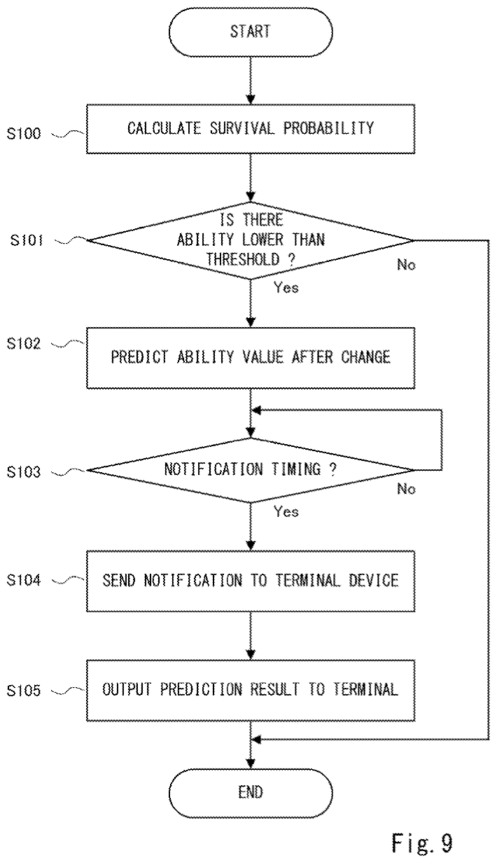

Next, a flow of an outputting operation performed by the rehabilitation work support apparatus 100 will be described. FIG. 9 is a flowchart showing an example of the outputting operation related to the prediction of a change in an ability value, performed by the rehabilitation work support apparatus 100. The flow of the outputting operation related to the prediction of a change in an ability value will be described hereinafter with reference to FIG. 9.

In step S100, the change prediction unit 103 calculates a survival probability for each ability value of each patient. That is, the change prediction unit 103 predicts whether or not an ability value has changed for each type of ability and for each patient.

Next, in a step S101, the change prediction unit 103 determines whether or not there is an ability (an item in the FIM) of which a survival probability calculated in the step S100 is lower than a predetermined threshold (e.g., 0.5). When there is no ability of which the survival probability is lower than the predetermined threshold, the process is finished. In contrast, when there is an ability of which the survival probability is lower than the predetermined threshold, the process proceeds to a step S102.

In a step S102, the ability value prediction unit 104 predicts an ability value after the change.

Next, in the step S103, the notification output unit 106 determines whether or not it becomes a predetermined notification timing. For example, the notification output unit 106 determines whether or not it becomes a predetermined time immediately before the start of rehabilitation for the patient for whom the notification is to be sent. When it becomes the predetermined notification timing, the process proceeds to a step S104.

In the step S104, the notification output unit 106 transmits the notification message to the terminal device 500.

When the user operates the terminal device 500, in a step S105, the change prediction output unit 105 transmits the results of the predictions made by the change prediction unit 103 and the ability value prediction unit 104 to the terminal device 500. That is, when the user inputs an instruction for displaying the information to an application running on the terminal device 500, the change prediction output unit 105 transmits the prediction results to the terminal device 500. As a result, the prediction results are displayed on the terminal device 500.

Note that although a series of operations in which the process in the step S105 is performed after the processes in the steps S103 to S104 is shown in the flowchart shown in the figure, the process in the step S105 can be performed at an arbitrary timing after the steps S101 and S102.

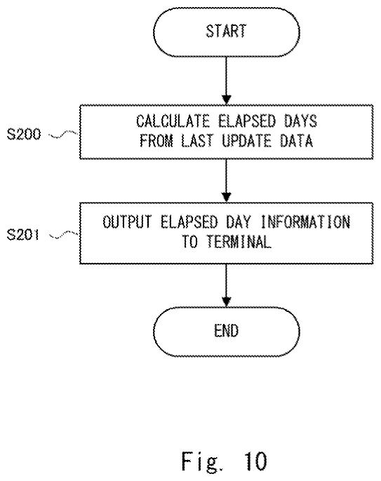

FIG. 10 is a flowchart showing an example of an outputting operation related to an un-updated period, performed by the rehabilitation work support apparatus 100. A flow of the outputting operation related to an un-updated period will be described hereinafter with reference to FIG. 10.

In a step S200, the elapsed time output unit 107 calculates the number of days elapsed from the last update date of an ability value based on the last update date of the ability value of each patient stored in the patient information storage unit 101 and the current date.

Then, when the user operates the terminal device 500, in a step S201, the elapsed time output unit 107 transmits information indicating the number of elapsed days calculated in the step S200 to the terminal device 500. As a result, the un-updated period for the ability value of each patient is displayed on the terminal device 500.

The rehabilitation work support system 10 according to the example embodiment has been described above. According to this system, when a prediction result indicting that an ability value of a given physical ability of a given patient has changed is obtained, it is sufficient if the ability value for this physical ability of this patient is evaluated. That is, according to the rehabilitation work support apparatus 100, a therapist can know, for which ability and for which patient, when he/she should evaluate an ability value. Therefore, it is possible to reduce the amount of work required to evaluate ability values. Accordingly, according to the rehabilitation work support apparatus 100, it is possible to efficiently manage patient's actual current ability values. Further, according to such a system, it becomes possible to keep track of the actual ability values at all times, so that the results (or effects) of rehabilitation is visualized (i.e., shown in a visual manner), thus contributing to increasing the motivations of a therapist and a patient. Further, since it becomes possible to keep track of the actual ability values at all times, it is possible to enable a patient to perform appropriate rehabilitation according to his/her actual ability values, thus contributing to the efficient recovery of the patient.

Note that the present invention is not limited to the above-described example embodiments and various modifications can be made within the scope and spirit of the invention.

Further, the whole or part of the example embodiments disclosed above can be described as, but not limited to, the following supplementary notes.

### (Supplementary Note 1)

A rehabilitation work support apparatus comprising:

change prediction means for predicting whether or not an actual current ability value of a patient during a rehabilitation period has changed from a latest ability value of that patient stored in a storage device based on an elapsed time from a time of an evaluation of the latest ability value of the patient stored in the storage device; and

change prediction output means for performing control so as to output a result of the prediction made by the change prediction means.

### (Supplementary Note 2)

The rehabilitation work support apparatus described in Supplementary note 1, wherein

the change prediction means makes the prediction for each type of ability, and

the change prediction output means outputs, as a result of the prediction, information indicating, for which ability, a current actual ability value of the patient has changed from a latest ability value of that patient stored in the storage device.

### (Supplementary Note 3)

The rehabilitation work support apparatus described in Supplementary note 1 or 2, wherein the change prediction means makes the prediction based on an elapsed time from a time of an evaluation of a latest ability value of the patient stored in the storage device and a feature of that patient.

### (Supplementary Note 4)

The rehabilitation work support apparatus described in Supplementary note 3, wherein the feature includes a content of rehabilitation performed by the patient.

### (Supplementary Note 5)

The rehabilitation work support apparatus described in Supplementary note 3 or 4, wherein the feature includes a latest ability value of the patient stored in the storage device.

### (Supplementary Note 6)

The rehabilitation work support apparatus described in any one of Supplementary notes 3 to 5, wherein

the change prediction means makes the prediction by performing a survival analysis in which a probability that an actual ability value of the patient having the feature at a specific time has not changed from a latest ability value of that patient stored in the storage device is defined as a survival probability, and

the specific time is a time representing an elapsed time from a time of an evaluation of the latest ability value of the patient stored in the storage device.

### (Supplementary Note 7)

The rehabilitation work support apparatus described in any one of Supplementary notes 1 to 6, further comprising ability value prediction means for predicting an ability value after a change by inputting target patient information into a prediction model using a plurality of pieces of past patient information, the target patient information being information about the patient during the rehabilitation period, and each of the plurality of pieces of past patient information being information about a respective one of a plurality of past patients who performed rehabilitation in a past, wherein

each of the pieces of past patient information is information including at least an ability value of a respective one of the past patients obtained before the change and that obtained after the change.

### (Supplementary Note 8)

The rehabilitation work support apparatus described in any one of Supplementary notes 1 to 7, further comprising elapsed time output means for performing control so as to output information representing, for each patient, an elapsed time from a time of a last update of an ability value stored in the storage device.

### (Supplementary Note 9)

The rehabilitation work support apparatus described in any one of Supplementary notes 1 to 8, further comprising notification output means for performing control so as to output a notification message when an actual current ability value of the patient during the rehabilitation period is predicted to have changed from a latest ability value of that patient stored in the storage device.

### (Supplementary Note 10)

The rehabilitation work support apparatus described in Supplementary note 9, wherein the notification output means performs control so as to output the notification message at a timing corresponding to a timing at which rehabilitation of the patient whose ability value is predicted to have changed is performed.

### (Supplementary Note 11)

The rehabilitation work support apparatus described in Supplementary note 9 or 10, wherein the notification output means performs control so as to output the notification message again when a predetermined operation has not been performed within a predetermined time after outputting the notification message.

### (Supplementary Note 12)

A rehabilitation work support system comprising: a rehabilitation work support apparatus; and a terminal device, wherein

the rehabilitation work support apparatus comprises:

change prediction means for predicting whether or not an actual current ability value of a patient during a rehabilitation period has changed from a latest ability value of that patient stored in a storage device based on an elapsed time from a time of an evaluation of the latest ability value of the patient stored in the storage device; and

change prediction output means for performing control so as to output a result of the prediction made by the change prediction means to the terminal device.

### (Supplementary Note 13)

The rehabilitation work support system described in Supplementary note 12, wherein

the change prediction means makes the prediction for each type of ability, and

the change prediction output means outputs, as a result of the prediction and to the terminal device, information indicating, for which ability, a current actual ability value of the patient has changed from a latest ability value of that patient stored in the storage device.

### (Supplementary Note 14)

A rehabilitation work support method comprising:

predicting whether or not an actual current ability value of a patient during a rehabilitation period has changed from a latest ability value of that patient stored in a storage device based on an elapsed time from a time of an evaluation of the latest ability value of the patient stored in the storage device; and

performing control so as to output a result of the prediction.

### (Supplementary Note 15)

A non-transitory computer readable medium storing a program for causing a computer to perform:

a change prediction step of predicting whether or not an actual current ability value of a patient during a rehabilitation period has changed from a latest ability value of that patient stored in a storage device based on an elapsed time from a time of an evaluation of the latest ability value of the patient stored in the storage device; and

a change prediction output step of performing control so as to output a result of the prediction made in the change prediction step.

Although the present invention is described above with reference to example embodiments, the present invention is not limited to the above-described example embodiments. Various modifications that can be understood by those skilled in the art can be made to the configuration and details of the present invention within the scope of the invention.

This application is based upon and claims the benefit of priority from Japanese patent application No. 2019-184151, filed on Oct. 4, 2019, the disclosure of which is incorporated herein in its entirety by reference.

### REFERENCE SIGNS LIST

1 REHABILITATION WORK SUPPORT APPARATUS
2 CHANGE PREDICTION UNIT
3 CHANGE PREDICTION OUTPUT UNIT
10 REHABILITATION WORK SUPPORT SYSTEM
50 CHOICE
100 REHABILITATION WORK SUPPORT APPARATUS
101 PATIENT INFORMATION STORAGE UNIT
102 PATIENT INFORMATION ACQUISITION UNIT
103 CHANGE PREDICTION UNIT
104 ABILITY VALUE PREDICTION UNIT
105 CHANGE PREDICTION OUTPUT UNIT
106 NOTIFICATION OUTPUT UNIT
107 ELAPSED TIME OUTPUT UNIT
150 NETWORK INTERFACE
151 MEMORY
152 PROCESSOR
400 NETWORK
500 TERMINAL APPARATUS
500A PORTABLE TERMINAL DEVICE
500B NON-PORTABLE TERMINAL DEVICE

What is claimed is:

1. A rehabilitation work support apparatus comprising:
at least one memory storing program instructions; and
at least one processor configured to execute the instructions stored in the memory to:
predict whether or not an actual current ability value of a patient during a rehabilitation period has changed from a latest ability value of that patient stored in a storage device based on an elapsed time from a time of an evaluation of the latest ability value of the patient stored in the storage device; and
perform control so as to output a result of the prediction.

2. The rehabilitation work support apparatus according to claim 1, wherein the processor is further configured to execute the instructions to:
make the prediction for each type of ability, and
output, as a result of the prediction, information indicating, for which ability, a current actual ability value of the patient has changed from a latest ability value of that patient stored in the storage device.

3. The rehabilitation work support apparatus according to claim 1, wherein the processor is further configured to execute the instructions to make the prediction based on an elapsed time from a time of an evaluation of a latest ability value of the patient stored in the storage device and a feature of that patient.

4. The rehabilitation work support apparatus according to claim 3, wherein the feature includes a content of rehabilitation performed by the patient.

5. The rehabilitation work support apparatus according to claim 3, wherein the feature includes a latest ability value of the patient stored in the storage device.

6. The rehabilitation work support apparatus according to claim 3, wherein
the processor is further configured to execute the instructions to make the prediction by performing a survival analysis in which a probability that an actual ability value of the patient having the feature at a specific time has not changed from a latest ability value of that patient stored in the storage device is defined as a survival probability, and
the specific time is a time representing an elapsed time from a time of an evaluation of the latest ability value of the patient stored in the storage device.

7. The rehabilitation work support apparatus according to claim 1, wherein
the processor is further configured to execute the instructions to predict an ability value after a change by inputting target patient information into a prediction model using a plurality of pieces of past patient information, the target patient information being information about the patient during the rehabilitation period, and each of the plurality of pieces of past patient information being information about a respective one of a plurality of past patients who performed rehabilitation in a past, and
each of the pieces of past patient information is information including at least an ability value of a respective one of the past patients obtained before the change and that obtained after the change.

8. The rehabilitation work support apparatus according to claim 1, wherein
the processor is further configured to execute the instructions to perform control so as to output information representing, for each patient, an elapsed time from a time of a last update of an ability value stored in the storage device.

9. The rehabilitation work support apparatus according to claim 1, wherein
the processor is further configured to execute the instructions to perform control so as to output a notification message when an actual current ability value of the patient during the rehabilitation period is predicted to have changed from a latest ability value of that patient stored in the storage device.

10. The rehabilitation work support apparatus according to claim 9, wherein the processor is further configured to execute the instructions to perform control so as to output the notification message at a timing corresponding to a timing at which rehabilitation of the patient whose ability value is predicted to have changed is performed.

11. The rehabilitation work support apparatus according to claim 9, wherein the processor is further configured to execute the instructions to perform control so as to output the notification message again when a predetermined operation has not been performed within a predetermined time after outputting the notification message.

12. (canceled)

13. (canceled)

14. A rehabilitation work support method comprising:
predicting whether or not an actual current ability value of a patient during a rehabilitation period has changed from a latest ability value of that patient stored in a storage device based on an elapsed time from a time of an evaluation of the latest ability value of the patient stored in the storage device; and
performing control so as to output a result of the prediction.

15. A non-transitory computer readable medium storing a program for causing a computer to perform:
a change prediction step of predicting whether or not an actual current ability value of a patient during a rehabilitation period has changed from a latest ability value of that patient stored in a storage device based on an elapsed time from a time of an evaluation of the latest ability value of the patient stored in the storage device; and
a change prediction output step of performing control so as to output a result of the prediction made in the change prediction step.
